(19) United States
(12) Patent Application Publication
(10) Pub. No.: US 2002/0192314 A1
(43) Pub. Date: Dec. 19, 2002

(54) Dietary supplement compositions

(76) Inventors: Suk H. Cho, Idaho Falls, ID (US); Lynn Perkes, Rexbunrg, MD (US)

Correspondence Address: RICHARD J. ANDERSON, Fish & Richardson P.C., P.A., Suite 3300, 60 South Sixth Street, Minneapolis, MN, 55402, US

(21) Appl. No.: 09/800,195

(22) Filed: Mar. 6, 2001

(51) Int. Cl. A61K 35/78

(52) U.S. Cl. 424/766

(57) ABSTRACT

The invention provides dietary supplements. Such dietary supplements can inhibit platelet aggregation or LDL cholesterol oxidation. Typically, a dietary supplement of the invention contains a combination of at least one grape skin extract and at least one grape seed extract. The invention also provides methods of inhibiting platelet aggregation or LDL cholesterol oxidation in a mammal.

### TECHNICAL FIELD

The invention relates to dietary supplements. Specifically, the invention relates to dietary supplements containing grape skin and grape seed extracts.

### BACKGROUND

Coronary artery disease, myocardial infarction, stroke, and other vascular occlusions are major health concerns. A common characteristic of these diseases is the atherosclerotic process, which is the narrowing of arteries. Blood platelets contribute to the development and progression of atherosclerosis by releasing growth factors, chemotactic substances, and other factors that accelerate the atherosclerotic process. In addition, platelet aggregation at or near the point of arterial damage contributes to the development of atherosclerosis and acute platelet thrombus formation. Low-density lipoprotein (LDL) cholesterol also is associated with atherosclerosis. It has been proposed that non-atherogenic LDL cholesterol circulating in the blood is converted to atherogenic LDL cholesterol through oxidation of polyunsaturated lipids, which leads to modification of the apoprotein.

Physicians use various drugs, such as aspirin, to treat atherosclerotic conditions. Aspirin, however, is not without negative side effects such as gastrointestinal irritation. Interventions such as angioplasty are available to dilate stenosed arteries and thereby increase blood flow. Interventional techniques, however, produce intimal and medial artery damage and expose thrombogenic surfaces. As such, re-stenosis and the incidence of sudden coronary death following angioplasty is a major concern for patients with known or suspected coronary artery disease. Given the grave consequences of atherosclerosis and the costs associated with medical treatments, there is a need for pharmacologic and nutritional interventions that are useful for preventing the occurrence and reoccurrence of these conditions.

### SUMMARY

The invention provides dietary supplements for inhibiting platelet aggregation or LDL cholesterol oxidation. Typically, dietary supplements of the invention include at least one grape skin extract and at least one grape seed extract. The extracts can have particular levels of polyphenols and/or flavonoids. In addition, the extracts can be combined at ratios that effectively inhibit platelet aggregation or LDL cholesterol oxidation. The invention also provides methods of inhibiting platelet aggregation or LDL cholesterol oxidation in a mammal as well as methods of treating conditions associated with platelet aggregation.

In general, the invention features a dietary supplement containing a grape skin extract and a Muscat variety grape seed extract. The ratio of the grape skin extract to the Muscat variety grape seed extract can be about 3 to 1, 4 to 1, or 5 to 1. The grape skin extract can contain at least about 25 percent polyphenolics. The grape skin extract can be a Zinfandel grape skin extract. The Muscat variety grape seed extract can contain at least about 70 percent polyphenolics. The Muscat variety grape seed extract can contain at least about 3.5 percent monomeric flavanols (e.g., at least about 7.5 percent monomeric flavanols or at least about 10 percent monomeric flavanols). The Muscat variety grape seed extract can contain at least about 60 percent oligomeric flavanols. The Muscat variety grape seed extract can contain less than about 35 percent polymeric flavanols. The Muscat variety grape seed extract can contain less than about 30 percent polymeric flavanols. The administration of the dietary supplement to a dog can inhibit platelet aggregation when measured with the Foltz model test, where the administration is 25 mg of the dietary supplement per kg of dog weight, and where the inhibition is measured at least 18 hours after the administration. The inhibition can be measured at least 24 hours after the administration. dietary supplement can contain ginkgo biloba, bilberry, quercetin, or enzyme (e.g., bromolein, papain, a fungal protease, an acid stable protease, a neutral stable protease, an alkaline stable protease, and mixtures thereof). The dietary supplement can inhibit platelet aggregation when measured with an in vitro platelet aggregometry test using 300 mg of the dietary supplement per liter of whole blood. The dietary supplement can inhibit LDL cholesterol oxidation when measured with a LDL oxidation test at a dosage of 10 mg of the dietary supplement per liter of cholesterol. The dietary supplement can be a pill, a powder, or a liquid. The Muscat variety of the Muscat variety grape seed extract can be unfermented.

In another embodiment, the invetion features a dietary supplement containing a grape skin extract and a grape seed extract, where the ratio of the grape skin extract to the grape seed extract is between 3 to 1 and 10 to 1. The ratio can be 3 to 1, 4 to 1, or 5 to 1. The grape seed extract can contain at least about 70 percent polyphenolics. The grape seed extract can be a Muscat variety grape seed extract.

Another embodiment of the invention features a dietary supplement containing a grape skin extract and a grape seed extract, where the grape seed extract contains at least about 70 percent polyphenolics.

Another embodiment of the invention features a dietary supplement containing a grape skin extract and a grape seed extract, where the grape seed extract contains at least about 3.5 percent monomeric flavanols, at least about 60 percent oligomeric flavanols, and less than about 35 percent polymeric flavanols.

In another aspect, the invention features a method of inhibiting platelet aggregation or LDL cholesterol oxidation in a mammal. The method includes administering a dietary supplement to the mammal, where the dietary supplement is selected from the group consisting of (a) a dietary supplement containing a grape skin extract and a Muscat variety grape seed extract, (b) a dietary supplement containing a grape skin extract and a grape seed extract, where the ratio of the grape skin extract to the grape seed extract is between 3 to 1 and 10 to 1, (c) a dietary supplement containing a grape skin extract and a grape seed extract, where the grape seed extract contains at least about 70 percent polyphenolics, and (d) a dietary supplement containing a grape skin extract and a grape seed extract, where the grape seed extract contains at least about 3.5 percent monomeric flavanols, at least about 60 percent oligomeric flavanols, and less than about 35 percent polymeric flavanols. The dietary supplement can be administered orally.

Unless otherwise defined, all technical and scientific terms used herein have the same meaning as commonly understood by one of ordinary skill in the art to which this invention pertains. Although methods and materials similar or equivalent to those described herein can be used in the practice or testing of the invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DETAILED DESCRIPTION

The invention provides dietary supplements that contain at least one grape skin extract and at least one grape seed extract. Such dietary supplements can inhibit platelet aggregation and LDL cholesterol oxidation in a mammal. A synergistic effect on the inhibition of platelet aggregation and LDL cholesterol oxidation was observed with dietary supplements containing grape extracts. The grape extracts used in dietary supplements of the invention can have particular levels of polyphenols or flavonoids. In addition, dietary supplements having particular ratios of grape skin extract to grape seed extract were found to increase inhibition of platelet aggregation and LDL cholesterol oxidation. While not bound by a particular theory of action, the combinations of grape skin extract and grape seed extract as described herein can act synergistically by increasing bioavailability or pharmacologic interactions to thereby inhibit blood platelet aggregation or LDL cholesterol oxidation.

Grape Skin Extract

One component in a dietary supplement of the invention is a grape skin extract. Extraction is a process whereby the desired constituents of a plant or plant part are removed using a solvent. To produce an extract, plant material is usually first cleaned and dried, if necessary. Drying can be done naturally (e.g., by air drying) or artificially (e.g., using warm-air fans or conveyor dryers). The plant material can then be ground, cut, or shredded using, for example, hammer action, pressure, friction, or impact cutting. Methods of removing the desired constituents from the plant material include, without limitation, organic solvent extraction, supercritical gas extraction, and steam distillation. The ability to use a number of different solutes, diluents, extractants, and aqueous phases as well as rapid extraction kinetics for many separations, makes solvent extraction a powerful separation method. By way of example, there are a number of procedures for organic solvent extraction, including maceration (soaking and agitating the plant material with a solvent), percolation (repeated rinsing of the plant material with a solvent), and countercurrent extraction (continuous flow of a solvent in the opposite direction as the plant material). Representative solvents include, without limitation, ethanol, benzene, toluene, and ether. Aqueous extracts, such as decoctions (produced by boiling the plant material such as hard tissues), infusions (produced by steeping the plant material such as soft tissues) or macerations, can also be produced. In addition, numerous other separation procedures can be used to further purify desired components or remove unwanted or contaminating components. Examples of such separation procedures include, without limitation, decanting, filtration, sedimentation, centrifugation, heating, adsorption, precipitation, chromatography, or ion exchange. The resulting products can be subsequently evaporated, vaporized, lyophilized, spray dried, freeze-dried, or vacuum dried. The Porter Assay (Porter et al., Phytochemistry, 25:223-230 (1986)) can be used to assess the quality and consistency of grape skin extracts.

To produce a grape skin extract useful in a dietary supplement of the invention, grape skin can be extracted from the pomace with an aqueous solution. The aqueous extract can be adsorbed in an organic column and desorbed with alcohol (e.g., ethanol), while the collected eluent can be spray or freeze dried. A grape skin extract used in the invention can be a solution or a soluble powder.

A grape skin extract can contain total polyphenols of at least about 25 percent (e.g., at least about 30, 40, 50, 60, 70, 80, 90, 95, or 100 percent) in gallic acid equivalents (Singleton et al., Am. J. Enol. Vitic, 16:144-58 (1965)). Typically, a grape skin extract contains at least about 0.1 percent anthocyanin (e.g., at least about 0.5, 1, 5, 10, 15, 20, or more percent anthocyanin). In addition, a grape skin extract can contain natural polysaccharides and/or polysaccharides added during post-extraction. Such polysaccharides can have any molecular weight. For example, a grape skin extract can contain a polysaccharide having a molecular weight greater than 500 daltons (e.g., malto-dextran). Typically, a grape skin extract contains from about 1 percent to about 50 percent polysaccharides based on dry weight.

Any color grape can be used to make a grape skin extract. For example, white grapes, red grapes, or mixtures of white and red grapes can be used to make a grape skin extract. In addition, any type of grape can be used to make a grape skin extract. For example, Muscat grapes (e.g., Muscat hamburg variety grapes), Colombard grapes, Chenin Blanc grapes, Gewurztraminer grapes, Zinfandel grapes, Cabernet Sauvignon grapes, Barbera grapes, Syrah grapes, or any mixture of Muscat grapes, Colombard grapes, Chenin Blanc grapes, Gewurztraminer grapes, Zinfandel grapes, Cabernet Sauvignon grapes, Barbera grapes, and Syrah grapes can be used to make a grape skin extract. Grape skins used to produce a grape skin extract of a dietary supplement can be unfermented. An unfermented grape skin extract can inhibit platelet aggregation or LDL cholesterol oxidation more effectively than a fermented grape skin extract of equal weight. A grape skin extract can be obtained from any source. For example, a grape skin extract can be obtained from a commercial vendor that sells grape skin extracts. Such vendors include Polyphenolics (Madera, Calif.) and Bio Serae (Montilieu, France).

Grape Seed Extract

Another component in a dietary supplement of the invention is an extract of grape seed. As described above, numerous methods can be used to produce an extract. To produce a grape seed extract useful in the invention, the grape seeds are usually first separated from the pomace. The seeds then can be extracted in a hydro-alcoholic solution (e.g., 20 percent to 60 percent ethanol in water (v/v)), and the resulting product directly spray or freeze dried. Alternatively, grape seeds can be extracted in either a hydro-alcoholic or an aqueous solution. The resulting product can be adsorbed in an organic column and desorbed with alcohol (e.g., ethanol), and the collected eluent can be dried. A grape seed extract can be a solution or a soluble powder.

A grape seed extract can contain total polyphenols of at least about 70 percent (e.g., at least about 75, 80, 85, 90, 95, or 100 percent) in gallic acid equivalents. A dietary supplement can contain a grape seed extract having any amount of monomeric flavanols. For example, a grape seed extract can contain 1 to 10 percent catechin and 1 to 10 percent epi-catechin. The term “monomeric flavanols” as used herein refers to single phenolic flavanols such as catechin, epi-catechin, gallates, gallocatechin, epiatachin, epigallo catechin, non-gallo catechins, and gallic esters. Typically, a grape seed extract of a dietary supplement contains at least about 3.5 percent monomeric flavanols (e.g., at least about 4, 5, 6, 7, 8, 9, 10, 11, 12, or more percent monomeric flavanols). A dietary supplement can contain a grape seed extract having any amount of oligomeric flavanols. The term “oligomeric flavanols” as used herein refers to dimers, trimers, tetramer, pentamer, hexamers, and/or heptamers of the above-described monomeric flavanols. Examples of oligomeric flavanols include, without limitation, proanthocyanidins. Typically, a grape seed extract of a dietary supplement contains at least about 60 percent oligomeric flavanols (e.g., at least about 65, 70, 75, 80, or more percent oligomeric flavanols). A dietary supplement can contain a grape seed extract having any amount of polymeric flavanols. The term “polymeric flavanols” as used herein refers to octamers and greater of the above-described monomeric flavanols. Typically, a grape seed extract of a dietary supplement contains less than about 35 percent polymeric flavanols (e.g., less than about 30, 25, 20, or less percent polymeric flavanols).

The monomeric, oligomeric, and polymeric content of a grape seed extract can be determined by, for example, HPLC analysis. Typically, the amount of proanthocyanin in a grape seed extract is greater than or equal to about 100 Procyanidolic Value Units (Bate-Smith, Phytochemistry, 12:1809-12 (1973)) and the amount of procyanin is greater than or equal to about 200 Porter Value Units (Porter et al., Phytochemistry, 25:223-230 (1986)).

Any color grape can be used to make a grape seed extract. For example, white grapes, red grapes, or mixtures of white and red grapes can be used to make a grape seed extract. In addition, any type of grape can be used to make a grape seed extract. For example, Muscat grapes (e.g., Muscat hamburg variety grapes), Colombard grapes, Chenin Blanc grapes, Gewurztraminer grapes, Zinfandel grapes, Cabernet Sauvignon grapes, Barbera grapes, Syrah grapes, or any mixture of Muscat grapes, Colombard grapes, Chenin Blanc grapes, Gewurztraminer grapes, Zinfandel grapes, Cabernet Sauvignon grapes, Barbera grapes, and Syrah grapes can be used to make a grape seed extract.

Grape seeds used to produce a grape seed extract of a dietary supplement can be uncrushed and/or unfermented. An unfermented grape seed extract can inhibit platelet aggregation or LDL cholesterol oxidation more effectively than a fermented grape seed extract of equal weight. A grape seed extract can be obtained from any source. For example, unfermented grape seed extracts are commercially available from Greenway International (Orem, Utah), Omega Biotech (Sidney, British Columbia), and Industrial Laboratories (Denver, Colo.).

Optional Components of a Dietary Supplement

A. Protein

A dietary supplement can contain one or more proteins. Proteins that are useful in combination with a grape skin extract and grape seed extract of the invention can be soluble in water or alcohol. For example, crude protein extracts, such as those from soy, whey, rice, pineapple, aloe, or papaya, heterogeneous classes of proteins such as albumin, enzymes such as proteases, or mixtures of such proteins are useful in combination with grape skin extracts and grape seed extracts to make dietary supplements of the invention. Representative enzymes that can be used in a dietary supplement of the invention include, without limitation, bromolein, papain, fungal proteases, acid stable proteases, neutral stable proteases, alkaline stable proteases, or mixtures thereof. Enzymes useful in the invention can be derived from porcine, bovine, fungi, or plants. Enzymes that can be used in the dietary supplements described herein are commercially available from, for example, National Enzyme Company (Forsyth, Mo.) or Novo Nordisk (Franklinton, N.C.) and a particular enzyme blend suitable for use in a dietary supplement of the invention is described in WO 99/07400.

B. Radical Scavengers, Antioxidants, or Reducing Agents

A dietary supplement can contain one or more radical scavengers, antioxidants, reducing agents, or mixtures thereof. Typically, a dietary supplement contains one or more radical scavengers, antioxidants, reducing agents, or mixtures thereof in an amount that effectively reduces oxidation or degradation of the grape skin extract and/or the grape seed extract. Examples of radical scavengers and antioxidants include, without limitation, ascorbic acid, tocopheryl acetate, tocopheryl palmitate, tocopherol, and butyl hydroxytoluene. Sodium bisulfite is an example of a reducing agent that can be incorporated into a dietary supplement.

C. Chelators

A dietary supplement can contain one or more chelators. A chelator can bind to contaminating heavy metals. Heavy metals can act as catalysts to degradation or oxidation of components within the grape skin extract and/or the grape seed extract. Examples of chelators include, without limitation, citric acid, soluble salts of citric acid, phosphates, nitrilotriacetic acid, soluble salts of nitrilotriacetic acid, sodium carboxymethyl oxymalonate, sodium carboxymethyl oxysuccinate, ethylendiaminetetracarboxylic acid, soluble salts of ethylendiaminetetracarboxylic acid, acrylic acid polymers, acrylic acid copolymers, methacrylic acid, and maleic acid.

D. Botanical Extracts and Flavanoids

A dietary supplement can contain botanical extracts (e.g., herbal extracts). Non-limiting examples of botanical extracts include those extracts from chamomile, rosemary, aloe, nettle, centella asiatica, ginkgo biloba, bilberry, apple, garlic powder, olive oil, and blueberry. Flavonoids (purified or from a botanical extract) also can be included in a dietary supplement of the invention. Flavonoids include, without limitation, citrus bioflavonoids and quercetin. Suppliers of the above-mentioned botanical extracts and flavonoids include, but are not limited to, Indena (Milan, Italy), Weinstein Nutritional (Irvine, Calif.), OptiPure (Los Angeles, Calif.) and Botanicals International (Long Beach, Calif.).

Formulations of a Dietary Supplement

The invention provides a dietary supplement containing at least one grape skin extract and at least one grape seed extract. The term “dietary supplement” as used herein refers to any composition that supplements the diet with one or more dietary ingredients (e.g., vitamins, minerals, amino acids, herbs, botanicals, concentrates, metabolites, extracts, or combination thereof). The ratio of grape skin extract to grape seed extract is a characteristic that can affect the amount of inhibition of platelet aggregation or LDL cholesterol oxidation. The ratio of grape skin extract to grape seed extract present in a dietary supplement of the invention can be a ratio that is between about 3 to 1 and about 10 to 1 (e.g., about three times, four times, or five times the amount of grape skin extract as compared to grape seed extract). The ratio is based on the dry weight of each extract. It is noted, however, that a dietary supplement can be made by mixing a grape skin extract with a grape seed extract or by adding the product from a single extraction performed using a mixture of grape skin and grape seed as the starting material. In addition, those of skill in the art will appreciate that a ratio of grape skin extract to grape seed extract of about 4 to 1 (4:1) includes 3.8:1, 3.9:1, 4.1:1, 4.2:1, 4:0.9, 4:1.1, and variations thereof.

Typically, dietary supplements are ingested. For example, a dietary supplement can be administered orally or intragastrically. In addition, dietary supplements can be administered by other routes such as nasally, intravenously, intramuscularly, subcutaneously, sublingually, intrathecally, or intradermally. The route of administration can depend on a variety of factors, such as the environment (e.g., the circumstances resulting in platelet aggregation) and therapeutic goals. As used herein, mammals generally refer to humans, but also can include domesticated mammals (e.g., dogs, cats, and livestock such as cows, horses, pigs, and sheep) in which inhibiting platelet aggregation or LDL cholesterol oxidation is desirable. Conditions under which inhibiting platelet aggregation or LDL cholesterol oxidation is desirable include, without limitation, atherosclerosis, coronary artery disease, myocardial infarction, femoral artery disease, vascular occlusion, angina pectoris, and during or after a stroke.

Any amount of a dietary supplement can be administered to a mammal. The dosages of a dietary supplement will depend on many factors including the mode of administration. Typically, the amount of grape skin extract and grape seed extract contained within a single dose of a dietary supplement will be an amount that effectively inhibits platelet aggregation without inducing significant toxicity. In particular, a dietary supplement of the invention can be formulated in a dose such that an individual receives from about 4 mg up to about 50 mg of grape seed extract per kg of body weight. Typically, a dietary supplement of the invention can be administered in an amount from about 5 mg up to about 500 mg per kg of body weight (e.g., 10 mg, 50 mg, 100 mg, or 250 mg).

By way of example, dietary supplements of the invention can be in the form of a liquid, solution, suspension, pill, capsule, tablet, gelcap, powder, gel, ointment, cream, nebulae, mist, atomized vapor, aerosol, or phytosome. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Dietary supplements of the invention also can contain pharmaceutically acceptable additives such as suspending agents, emulsifying agents, non-aqueous vehicles, preservatives, buffer salts, flavoring, coloring, and sweetening agents as appropriate. Preparations for oral administration also can be suitably formulated to give controlled release of the active ingredients.

In addition, dietary supplements of the invention can contain a pharmaceutically acceptable carrier for administration to a mammal, including, without limitation, sterile aqueous, or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration.

Methods to Evaluate Platelet Aggregation and LDL Cholesterol Oxidation

Platelet aggregation and LDL cholesterol oxidation can be examined using a number of methods known to those of skill in the art. The Folts model test for evaluating platelet aggregation, a platelet aggregometry test, and a method for determining LDL cholesterol oxidation are described in detail in WO 99/07400.

A brief description of the Folts model follows. A flow probe is placed on the coronary artery of an animal to measure blood flow, and the artery is clamped to create intimal and medial damage. A cylinder is placed around the artery at the site of damage to regulate constriction (e.g., the extent of stenosis). Platelet aggregation at the site of the damage can result in an increase in arterial pressure. The increase in arterial pressure can subsequently dislodge the aggregate and thereby reduce the arterial pressure. The effect on arterial pressure due to multiple cycles of aggregation and removal are termed cyclical flow reductions (CFRs). CFRs can be measured in the presence and absence of a dietary supplement (e.g., administered intravenously to the animal) and used as an indication of the platelet aggregation inhibitory capabilities of the dietary supplement. Thus, the Folts model test can be used to identify or evaluate platelet inhibitors, the extent of inhibitor activity, the effective dosages of inhibitors, the duration of inhibition, and the ability of an inhibitor to counteract platelet agonists.

An in vitro or ex vivo platelet aggregometry test can be performed to evaluate platelet aggregation. Briefly, in an ex vivo aggregometry test, a blood sample is drawn by standard methods, and the electrical resistance of the blood is determined (i.e., measured as a change in impedence). The electrical resistance of blood is normally low due to numerous ions and electrolytes present. A known platelet aggregation stimulus, such as adenosine diphosphate (ADP) or collagen, is then added to the blood sample to activate the platelets. Activated platelets adhere to the electrodes and the electrical resistance of the blood sample usually increases in a sigmoidal fashion as platelets aggregate on the electrodes. If a subject is administered a dietary supplement that inhibits platelet aggregation, the increase in resistance following the addition of a platelet stimulus is reduced. In an in vitro platelet aggregometry test, whole blood samples are treated directly in a tube or dish with, for example, a dietary supplement. The electrical resistance of the blood is then measured and used to evaluate the platelet inhibitory characteristics of such a dietary supplement.

The antioxidant properties of a dietary supplement can be measured using an assay for LDL cholesterol oxidation. Briefly, blood samples are drawn and LDL cholesterol is isolated. The isolated LDL cholesterol is combined with a dietary supplement to be evaluated, and the time (i.e., lag time) before cholesterol oxidation is observed is measured. Copper ions accelerate the production of conjugated dienes due to oxidation, while antioxidants prolong the onset of diene production. Since dienes absorb light at 234 nm, LDL cholesterol oxidation can be monitored by examining the absorbance at 234 nm as a function of time. Thus, the antioxidant properties of, for example, a dietary supplement or other compound can be evaluated by determining the time elapsed before LDL cholesterol oxidation is observed.

The invention will be further described in the following examples, which do not limit the scope of the invention described in the claims.

### EXAMPLES

### Example 1

### HPLC Analysis of Grape Seed Extract

For use as standards, 25.75 mg (25 ppm) gallic acid (97% purity), 102.00 mg (100 ppm) catechin (98% purity), and 101.00 mg (100 ppm) epicatechin gallate (99% purity) were dissolved in a small amount of methanol and brought up to 100 ml with water to produce a stock solution. 5 ml of the stock solution was then diluted into 45 ml of water to produce HPLC standards. Both the stock and diluted standards were stored at 4° C. The standards were run on each HPLC, and the peak areas for gallic acid, catechin, and epicatechin were very similar run to run (within 2.5%). Prior to HPLC, the samples and standards were centrifuged at 14,000 rpm for 10 minutes.

Mobile phases were prepared according to the following: Phase A contained 2% acetic acid (980 ml of HPLC grade water and 20 ml acetic acid); and Phase B contained 80% acetonitrile, 0.4% acetic acid (800 ml acetonitrile and 200 ml of Phase A). The mobile phases were filtered through a 0.45 μm HVLP-type filter. HPLC was performed using a Waters (Milford, Mass.) 996 Photodiode Array Detector and a Waters 510 Pump. HPLC conditions were as follows: 25 μl of sample or standard was injected into a Phenomenex P/NO 006-4097-EO, type Prodigy 5 μl ODS (3) 100A, 250×4.6 mm column or a Phenomenex P/NO 03A-4097-EO, type Prodigy 5 μl ODS (3) 100A, 30×4.6 mm guard column and run at 30° C. at a flow rate of 1.0 ml/min. The detection wavelength was 280 nm for a peak area of 10-84 minutes. The particular HPLC gradient used is shown in Table 1.

TABLE 1
HPLC gradient

| Time (min) | % A | % B | Curve |
|---|---|---|---|
| 0.00 | 100 | 0 | — |
| 3.00 | 100 | 0 | 6 |
| 6.00 | 96 | 4 | 6 |
| 15.00 | 90 | 4 | 6 |
| 30.00 | 85 | 15 | 6 |
| 50.00 | 77 | 23 | 6 |
| 60.00 | 75 | 25 | 6 |
| 66.00 | 70 | 30 | 6 |
| 80.00 | 50 | 50 | 6 |
| 83.00 | 20 | 80 | 6 |
| 85.00 | 100 | 0 | 6 |
| 105.00 | 100 | 0 | 6 |
| 110.00 | 100 | 0 | 6 |

Millinneum Software from Waters was used for integration of the peaks to determine relative polyphenol profiles of the extracts. The percent monomers was determined by the following formula:

(peak areas corresponding to gallic acid+catechin+epicatechin+epicatechin gallate)/total peak area;

the percent oligomers was determined using the following formula:

(peak area 10 to 67 minutes−the monomer peak area above)/total peak area;

and the percent polymers was determined using the following:

(peak are from 67 to 84 minutes)/total peak area.

Based on numerous repetitions, the area on the chromatogram from 84-94 minutes was attributed to solvent artifact.

### Example 2

### Platelet Aggregation in the Presence of Grape Skin Extract or Grape Seed Extract

Grape skin extracts and grape seed extracts were evaluated using an in vitro assay for platelet aggregation (whole blood platelet aggregometry). Three concentrations of either the grape skin extract or grape seed extract were prepared (25, 62.5, or 125 mg dissolved in 300 μl of DMSO and 700 μl of preservative-free saline). 4 μl of each solution was incubated for 5 min in 1 ml of whole blood (which had been diluted with an equal volume of preservative free saline; final blood concentrations were 200, 500 and 1000 mg/L). The blood was continuously stirred at 37° C. After the incubation period, collagen (2 mg/L) was added to the blood to induce platelet aggregation. A change in impedance was measured 7 min after the addition of collagen and represents the extent of platelet aggregation (i.e., the platelet response). As shown in Tables 2 and 3, there were several grape skin extracts and grape seed extracts, respectively, that inhibited platelet aggregation.

TABLE 2
Grape Skin Extracts

| Sample | | Total | | mg/L of extract to inhibit |
|---|---|---|---|---|
| ID | Grape Source | Phenols* | Extraction method | 50% platelet aggregation |
| 151 | Zinfandel | 25% | Water | 550 |
| 108 | Muscat hamburg | 80% | Hydro-alcoholic | Minimal activity |
| 109 | Muscat hamburg | 80% | Hydro-alcoholic | Minimal activity |
| 158-25 | Zinfandel | 25% | Water | 650 |
| 158-80 | Zinfandel | 80% | Water | 380 |

*in gallic acid equivalents

Grape skin extracts designated 151, 158-25, and 158-80 inhibited platelet aggregation compared to the other extracts examined. Samples 151, 158-25, and 158-80 are grape skin extracts produced from Zinfandel grapes using an aqueous extraction method.

TABLE 3
Grape seed extracts

| | | | mg/L of extract to |
|---|---|---|---|
| Sample | | | inhibit 50% platelet |
| ID | Grape Source | Extraction method | aggregation |
| 103 | Muscat hamburg | Hydro-alcoholic | 110 |
| 107 | Mixture of red and | Acetic acid/isopropanol/ethanol/water | 310 |
| | white* | | |
| 17 | Mixture of white* | Acetone/ethylacetate/isopropanol/ethanol | 109 |
| 136 | Muscat hamburg | Hydro-alcoholic | 120 |
| 156 | Muscat hamburg | Hydro-alcoholic | 110 |
| 165 A | Champagne | Proprietary non-ethanol/water extraction | 320 |
| | | (Indena) | |
| 165 B | Champagne | Proprietary non-ethanol/water extraction | >1200 |
| | | (Indena) | |
| 165 C | Mixture of white* | Water | 180 |
| 165 D | Mixture of red* | Water | 220 |
| 121 | Mixture of red and | Water | 300 |
| | white* | | |

*White grape mixture includes Chardonnay, Colombard, Chenin Blanc, and Zinfandel grapes; Red grape mixture includes Ruby Red grapes; Red and white mixtures are combinations of the white grape and red grape mixtures.

Grape seed extracts designated 103, 17, 136, and 156 demonstrated greater platelet inhibiting activity compared to the other extracts examined. Samples 103, 136 and 156 are grape seed extracts produced using hydro-alcoholic extraction and are from the Muscat hamburg variety of grapes.

Table 4 describes characteristics of the grape seed extracts that demonstrated greater platelet inhibiting activity as provided by the manufacturer.

TABLE 4
Grape seed extract analysis

| | | | Procyanol- | Mono- | Oligo- | Poly- |
|---|---|---|---|---|---|---|
| Sample | Total | Porter | dolic | mers | mers | mers |
| ID | Phenols (%) | Value | Value | (%) | (%) | (%) |
| 103 | 86.7 | 318 | 165 | 12.1 | 63 | 25 |
| 107 | 65.3 | 234 | 143 | 3.1 | 56 | 40.1 |
| 136 | 87.3 | 277.4 | 159.7 | 7.5 | 62.9 | 29.6 |
| 121 | 72.2 | 224 | 112 | 3.6 | 56.9 | 39.5 |

### Example 3

### LDL Oxidation in the Presence of Grape Seed Extract

LDL cholesterol oxidation is usually determined by measuring absorbance at 234 nm (AbS_{234 nm}) as a function of time under oxidative conditions. The antioxidant effectiveness of various grape seed extracts was determined. LDL cholesterol was prepared from human volunteer blood samples as described above. Isolated LDL cholesterol was then mixed with a buffer, vitamin E, or grape seed extract. Copper ions (5 μmole/L final concentration) were added to each sample. Grape seed extracts were prepared at 0.5 or 1.0 mg/L final concentration. The concentrations of grape seed extracts used were based on an estimate of expected blood levels of a dietary supplement based upon accepted blood absorption models. The amount of vitamin E used was comparable to the amount anticipated to be in the blood of a person administered 400 IU of vitamin E.

LDL cholesterol did not demonstrate appreciable oxidation until 45 min following the addition of copper ions. At the concentration used, vitamin E protected LDL cholesterol from oxidation for about 125 min. Grape seed extracts designated 107, 17, and 156 protected LDL cholesterol against oxidation for more than 150 min, for about 190 minutes, and for more than 225 minutes, respectively. At a concentration of 0.5 mg/L, all the grape seed extracts examined were better at protecting LDL cholesterol against oxidation than vitamin E (Table 5).

TABLE 5
LDL oxidation

| Sample | LDL oxidation lag time |
|---|---|
| Control | 70 min |
| Vitamin E | 125 min |
| 156 | 230 min |
| 107 | 155 min |
| 17 | 190 min |

### Example 4

### Synergy Between Grape Seed Extract and Grape Skin Extract

Grape seed extract (Sample 156) and grape skin extract (Sample 151) were used to examine platelet aggregation using whole blood in an in vitro platelet aggregometry. Platelet aggregation experiments with grape skin extract or grape seed extract alone indicated that the grape seed extract was about 5 times more potent than grape skin extract. Thus, the grape skin extract to grape seed extract ratio used was 5 to 1.

Grape skin extract (25 mg or 125 mg) or grape seed extract (12.0 mg or 62.5 mg) was dissolved in 300 μl of DMSO and 700 μl of preservative free saline. 4 μl of each solution was incubated for 5 min in 1 ml of whole blood (from human volunteers (n=11)) that had been diluted with an equal volume of preservative free saline to a final blood concentration of 100 or 500 mg/L. The platelet aggregation experiments were also performed using 100 mg/L grape seed extract+500 mg/L grape skin extract or 50 mg/L grape seed extract+250 mg/L grape skin extract. The blood was continuously stirred at 37° C. After a 5 min incubation period, collagen (2 mg/L) was added to the blood to induce platelet aggregation. The change in impedance was measured 7 min after the addition of collagen and represents the extent of platelet aggregation (i.e., the platelet response). The results are presented in Table 6.

TABLE 6
Inhibition of Platelet Aggregation

| Extract | % of Baseline Platelet Activity |
|---|---|
| No extract | 100% |
| Grape seed extract (100 mg/L) | 90% (p < 0.01) |
| Grape seed extract (50 mg/L) | 105% |
| Grape skin extract (500 mg/L) | 101% |
| Grape skin extract (250 mg/L) | 115% |
| Grape seed extract (100 mg/L) + | 30% (p < 0.001) |
| Grape skin extract (500 mg/L) | |
| Grape seed extract (50 mg/L) + | 80% (p < 0.005) |
| Grape skin extract (250 mg/L) | |

When the higher dose of grape skin and seed extracts were incubated together, a significant amount of platelet inhibition was observed (70% inhibition, p<0.001), indicating synergy between the grape skin and grape seed extracts. When the lower dose of grape skin and seed extracts were incubated together, a 20% inhibition was observed (p<0.005). No significant platelet inhibition was observed with the lower dose of either extract individually, and at the higher dose, only the grape seed extract showed a slight platelet inhibition (10% inhibition, p<0.01).

### Example 5

### Effect of Enzyme Blend on Grape Extracts

The effect of a proprietary enzyme blend (a combination of fungal and plant proteases) on the potency of the extracts was examined. The enzyme blend is thought to enhance the bioavailability of compounds. Seven male hound dogs were restricted from all medications and all know anti-platelet compounds for 14 days prior to the start of the study. After this “washout” period, a blood sample was drawn from the cephalic vein using a 19G butterfly needle and placed into an anti-coagulent (9 ml of blood into 1 ml of 3.9% sodium citrate). No pre-medication was administered prior to the blood draw. Whole blood platelet aggregation studies were performed using collagen (1 mg/L) as the platelet agonist.

The dogs were then randomly assigned one of the following daily oral treatments for eight days: Grape seed extract (Sample 156) (5 mg/kg); Grape skin extract (Sample 151) (20 mg/kg); Grape seed extract (5 mg/kg)+Grape skin extract (20 mg/kg); Grape seed extract (5 mg/kg)+Grape skin extract (20 mg/kg)+enzyme blend (2 mg/kg); Grape seed extract (5 mg/kg)+Grape skin extract (20 mg/kg)+enzyme blend (5 mg/kg); and Enzyme blend (5 mg/kg).

On the eighth day, blood sampling was repeated and platelet aggregation measurements were conducted as described above. The dogs were then “washed” out for 14 days and assigned another of the treatments described above. After eight days of treatment, blood sampling and aggregation measurements were repeated. The dogs were again “washed” out for 14 days and assigned yet another treatment. Aggregation experiments were repeated until measurements were obtained for each treatment in each dog.

TABLE 7
Inhibition of Platelet Aggregation

| Extract | % of Baseline Platelet Activity |
|---|---|
| No extract | 100% |
| Grape seed extract (5 mg/kg) | 100% |
| Grape skin extract (20 mg/kg) | 100% |
| Grape seed extract (5 mg/kg) + | 70% (p < 0.04) |
| Grape skin extract (20 mg/kg) | |
| Grape seed extract (5 mg/kg) + | 40% (p < 0.005) |
| Grape skin extract (20 mg/kg) + | |
| Enzyme blend (2 mg/kg) | |
| Grape seed extract (5 mg/kg) + | 40% (p < 0.007) |
| Grape skin extract (20 mg/kg) + | |
| Enzyme blend (5 mg/kg) | |
| Enzyme blend (5 mg/kg) | 100% |

When the grape skin extracts and the grape seed extracts were incubated together, a significant amount of platelet inhibition was observed (30% inhibition, p<0.04). When an enzyme blend was added to the grape seed and skin extracts, a 60% inhibition was observed (p<0.005 or p<0.007 depending upon the amount of enzyme blend added). No significant platelet inhibition was observed with either extract individually or with the enzyme blend alone. In addition, the dogs were studies 24 hours after a dietary supplement containing grape seed extract (5 mg/kg), grape skin extract (20 mg/kg), and enzyme blend (5 mg/kg) had been administered. Platelet aggregation was still decreased (50%±14%, p<0.05), demonstrating an inhibitory effect of the dietary supplement lasting at least 24 hours post-administration.

### Example 6

### Formulation of a Dietary Supplement

A dietary supplement of the invention can be made to contain the following ingredients:

| Ingredient | mg |
|---|---|
| Grape skin extract | 1460 |
| Muscat grape seed extract | 365 |
| Ginkgo biloba extract | 10 |
| Bilberry extract | 10 |
| Quercetin | 10 |
| Enzyme blend | 144 |

The above formulation is based upon a dose suitable for administration to an individual weighing about 160 pounds. Alternatively, the ginkgo biloba extract and/or the bilberry extract can be formulated in a dietary supplement at 1 mg each.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A dietary supplement comprising a grape skin extract and a Muscat variety grape seed extract.

2. The dietary supplement of claim 1, wherein the ratio of said grape skin extract to said Muscat variety grape seed extract is about 3 to 1.

3. The dietary supplement of claim 1, wherein the ratio of said grape skin extract to said Muscat variety grape seed extract is about 4 to 1.

4. The dietary supplement of claim 1, wherein the ratio of said grape skin extract to said Muscat variety grape seed extract is about 5 to 1.

5. The dietary supplement of claim 1, wherein said grape skin extract comprises at least about 25 percent polyphenolics.

6. The dietary supplement of claim 1, wherein said grape skin extract is a Zinfandel grape skin extract.

7. The dietary supplement of claim 1, wherein said Muscat variety grape seed extract comprises at least about 70 percent polyphenolics.

8. The dietary supplement of claim 1, wherein said Muscat variety grape seed extract comprises at least about 3.5 percent monomeric flavanols.

9. The dietary supplement of claim 1, wherein said Muscat variety grape seed extract comprises at least about 7.5 percent monomeric flavanols.

10. The dietary supplement of claim 1, wherein said Muscat variety grape seed extract comprises at least about 10 percent monomeric flavanols.

11. The dietary supplement of claim 1, wherein said Muscat variety grape seed extract comprises at least about 60 percent oligomeric flavanols.

12. The dietary supplement of claim 1, wherein said Muscat variety grape seed extract comprises less than about 35 percent polymeric flavanols.

13. The dietary supplement of claim 1, wherein said Muscat variety grape seed extract comprises less than about 30 percent polymeric flavanols.

14. The dietary supplement of claim 1, wherein administration of said dietary supplement to a dog inhibits platelet aggregation when measured with the Foltz model test, wherein said administration is 25 mg of said dietary supplement per kg of dog weight, and wherein said inhibition is measured at least 18 hours after said administration.

15. The dietary supplement of claim 14, wherein said inhibition is measured at least 24 hours after said administration.

16. The dietary supplement of claim 1, wherein said dietary supplement comprises ginkgo biloba.

17. The dietary supplement of claim 1, wherein said dietary supplement comprises bilberry.

18. The dietary supplement of claim 1, wherein said dietary supplement comprises quercetin.

19. The dietary supplement of claim 1, wherein said dietary supplement comprises an enzyme.

20. The dietary supplement of claim 19, wherein said enzyme is selected from the group consisting of bromolein, papain, a fungal protease, an acid stable protease, a neutral stable protease, an alkaline stable protease, and mixtures thereof.

21. The dietary supplement of claim 1, wherein said dietary supplement inhibits platelet aggregation when measured with an in vitro platelet aggregometry test using 300 mg of said dietary supplement per liter of whole blood.

22. The dietary supplement of claim 1, wherein said dietary supplement inhibits LDL cholesterol oxidation when measured with a LDL oxidation test at a dosage of 10 mg of said dietary supplement per liter of cholesterol.

23. The dietary supplement of claim 1, wherein said dietary supplement is a pill, a powder, or a liquid.

24. The dietary supplement of claim 1, wherein the Muscat variety of said Muscat variety grape seed extract is unfermented.

25. A dietary supplement comprising a grape skin extract and a grape seed extract, wherein the ratio of said grape skin extract to said grape seed extract is between 3 to 1 and 10 to 1.

26. The dietary supplement of claim 25, wherein said ratio is 3 to 1.

27. The dietary supplement of claim 25, wherein said ratio is 4 to 1.

28. The dietary supplement of claim 25, wherein said ratio is 5 to 1.

29. The dietary supplement of claim 25, wherein said grape seed extract comprises at least about 70 percent polyphenolics.

30. The dietary supplement of claim 25, wherein said grape seed extract is a Muscat variety grape seed extract.

31. A dietary supplement comprising a grape skin extract and a grape seed extract, wherein said grape seed extract comprises at least about 70 percent polyphenolics.

32. A dietary supplement comprising a grape skin extract and a grape seed extract, wherein said grape seed extract comprises at least about 3.5 percent monomeric flavanols, at least about 60 percent oligomeric flavanols, and less than about 35 percent polymeric flavanols.

33. A method of inhibiting platelet aggregation or LDL cholesterol oxidation in a mammal, said method comprising administering a dietary supplement to said mammal, wherein said dietary supplement is selected from the group consisting of:
(a) a dietary supplement comprising a grape skin extract and a Muscat variety grape seed extract,
(b) a dietary supplement comprising a grape skin extract and a grape seed extract, wherein the ratio of said grape skin extract to said grape seed extract is between 3 to 1 and 10 to 1,
(c) a dietary supplement comprising a grape skin extract and a grape seed extract, wherein said grape seed extract comprises at least about 70 percent polyphenolics, and
(d) a dietary supplement comprising a grape skin extract and a grape seed extract, wherein said grape seed extract comprises at least about 3.5 percent monomeric flavanols, at least about 60 percent oligomeric flavanols, and less than about 35 percent polymeric flavanols.

34. The method of claim 33, wherein said dietary supplement is administered orally.
